(19) United States
(12) Patent Application Publication
(10) Pub. No.: US 2014/0163542 A1
(43) Pub. Date: Jun. 12, 2014

(54) SYSTEM AND PROCEDURE FOR ENHANCING OCULAR DRAINAGE

(71) Applicant: Alcon Research, Ltd., Fort Worth, TX (US)

(72) Inventor: Guido Vezzu, Pfungen (CH)

(73) Assignee: Alcon Research, Ltd., Fort Worth, TX (US)

(21) Appl. No.: 14/065,057

(22) Filed: Oct. 28, 2013

Related U.S. Application Data
(60) Provisional application No. 61/735,772, filed on Dec. 11, 2012.

(51) Int. Cl. A61F 9/007

(52) U.S. Cl. CPC A61F 9/0079; A61F 9/00781
USPC 606/33

(57) ABSTRACT

Various systems, apparatuses, and processes may be used for enhancing ocular drainage. In particular implementations, a system for ocular drainage may include an ocular interface that includes a base and a number of diathermy needles. The diathermy needles may extend from the base a distance sufficient to penetrate a sclera of an eye and provide radio frequency energy to a trabecular meshwork.

### CROSS REFERENCE TO RELATED APPLICATIONS

This application claims the benefit of U.S. Provisional Application No. 61/735,772, filed Dec. 11, 2012, the entire contents of which are incorporated herein by reference.

### BACKGROUND

The present disclosure relates to ocular procedures, and more specifically to procedures for ocular drainage.

The human eye, in simple terms, functions to provide vision by transmitting and refracting light through a clear outer portion called the cornea and focusing the image by way of the lens onto the retina at the back of the eye. A number of nerve fibers, known as the optic nerve, carry visual information from the retina to the brain. The quality of the focused image depends on many factors including the size, shape, and length of the eye, and the shape and transparency of the cornea and lens.

Glaucoma is an eye disease in which the optic nerve is damaged. This can permanently damage vision in the affected eye(s) and lead to blindness if left untreated. Glaucoma is normally associated with increased fluid pressure in the eye(s).

Various procedures may be used to reduce the fluid pressure. For example, a plate glaucoma drainage device (GDD), such as an Ahmed valve or a Baerveldt tube shunt, may be implanted. In a typical plate GDD procedure, a plate is placed into the eye's subconjunctival space and a tube, which connects to the plate at one end, is fed anteriorly along the outside of the sclera and then inserted into the anterior chamber of the eye, where it permanently resides between the cornea and the iris. The implant aims to reduce intraocular pressure (IOP) in the treatment of glaucoma by bypassing the conventional, highly-resistive aqueous humor pathway (i.e., through the trabecular meshwork and into Schlemm's canal). Another procedure actually involves slicing through the sclera (e.g., with a knife) and cutting holes in the patient's trabecular meshwork (e.g., with scissors) to allow the aqueous humor to drain more readily to Schlemm's canal and into the lymphatic system.

### BRIEF SUMMARY

In one general implementation, a system for enhancing ocular drainage may include an ocular interface that includes a base and a number of diathermy needles extending from the base. The needles may extend a distance sufficient to penetrate a sclera of an eye and provide radio frequency energy to a trabecular meshwork therein.

In certain implementations, the base is an annular ring that is sized to fit around a cornea. Additionally, the diathermy needles may include an outer layer of insulation, such as in the form of an insulating sleeve, that extends from the end of the needles near the base to close to the distal end of the needles.

The system may also include an electrical signal source coupled to the ocular interface and adapted to apply a high frequency radio signal to the diathermy needles. The electrical signal may be adapted to cause the trabecular meshwork in the vicinity of the needles to contract, which causes a large portion of the trabecular meshwork to stretch.

Various implementations may include one or more features. For example, as opposed to prior art procedures, IOP may be treated without having to cut open portions of a patient's eye (e.g., to access the trabecular meshwork). In fact, the thickness of the diathermy needles may qualify the system for minimal invasive treatment of glaucoma. With help of such device, the trabecular meshwork could be coagulated at a sufficient number of locations to provoke many local meshwork contractions. Overall, the contractions results in stretching of the meshwork, which leads to a reduction of its flow resistance. Therefore, an improved flow into the Schlemm's canal and a corresponding lowered IOP can be expected.

The details and features of various implementations will be conveyed by the following description, along with the drawings.

### BRIEF DESCRIPTION OF THE SEVERAL VIEWS OF THE DRAWINGS

FIG. 1 illustrates an example system for enhancing ocular drainage.

FIGS. 2A-C illustrate an example system for enhancing ocular drainage in use.

FIG. 3 is a flowchart illustrating an example process for enhancing ocular drainage.

FIG. 4 illustrates and example needle having an outer layer of insulation.

FIG. 5 illustrates an example spacer.

### DETAILED DESCRIPTION

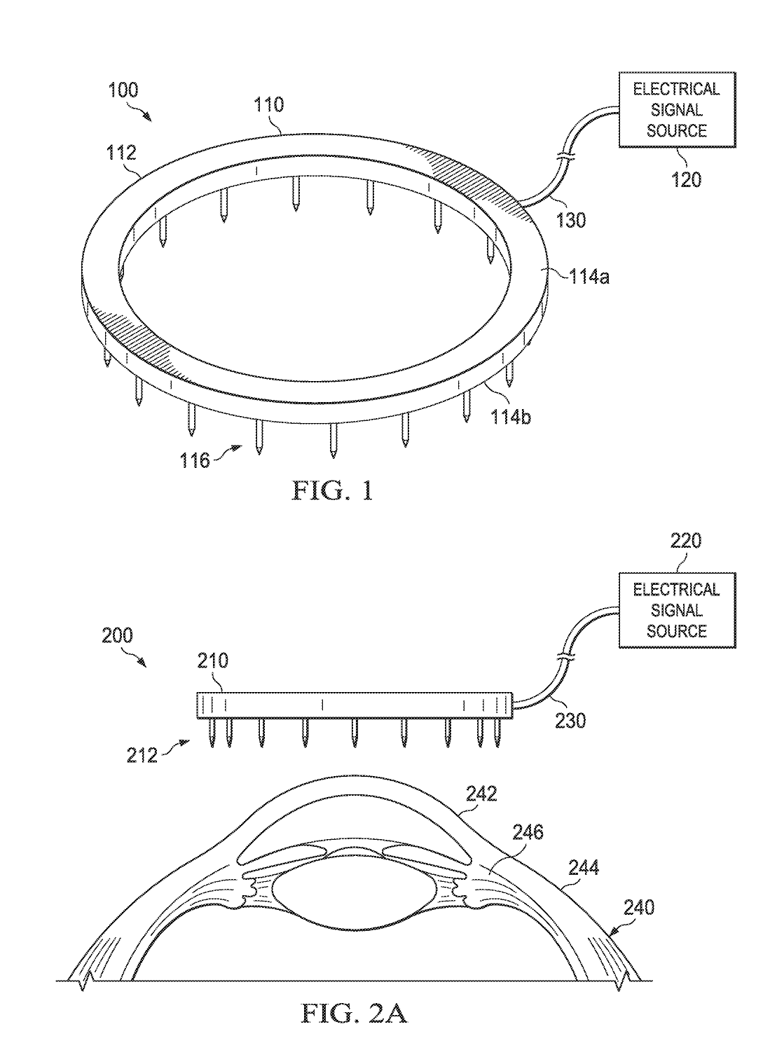

FIG. 1 illustrates an example system 100 for enhancing ocular drainage. System 100 includes an ocular interface 110 and an electrical signal source 120.

Ocular interface 110 includes a base 112 having a first surface 114a and a second surface 114b. In the illustrated implementation, base 112 is shaped as an annular ring. In other implementations, base 112 may have other shapes (e.g., oval). The inner diameter of base 112 may generally be sized to fit around a cornea of an eye. Thus, second surface 114b may contact an eye's sclera. In particular implementations, second surface 114b may contact an eye at the limbal contour. Base 112 may be made from metal, plastic, or any other appropriate material. In some implementations, the outside of base 112 is made of an inert and non-toxic material while the inside of base 112 may be made of another material. In particular implementations, the outer surface of base 112 may be made of plastic while the inner portion is made of a conducting metal.

Ocular interface 110 also includes a number of diathermy needles 116 that extend from second surface 114b. The tips of diathermy needles 116 may be adapted to apply radio frequency (RF) signals to eye tissue in their vicinity. Diathermy needles 116 may, for example, be between 0.01 mm and 0.1 mm in diameter and be made of metal or any other appropriate material. In some implementations, diathermy needles 116 may be monopolar. In certain implementations, diathermy needles 116 may be bipolar.

Diathermy needles 116 may extend far enough from base 112 to allow sufficient penetration into an eye for a far distance coagulation reaction to happen in the eye's trabecular meshwork. That is, in operation, the diathermy needles 116 are operable to produce coagulation within the eye at a location offset from the distal ends thereof. Thus, the diathermy needles 116 may extend sufficiently into the eye in order to produce coagulation at a desired depth therewithin. The location offset from a distal end of a diathermy needle 116 where coagulation is induced may be referred to as an interaction point. When an electrical signal is supplied to the diathermy needles 116, such as the signals described below, electromagnetic waves are emitted from the distal ends thereof and cause coagulation within tissues at the interaction point. The reaction may, for example, happen if the tips of the needles are in the trabecular meshwork itself or in the sclera. The coagulation effect of the needles typically manifests at a small distance from the tips. In particular implementations, needles 116 may extend far enough from base 112 to penetrate between approximately 0.8 to 1.2 mm into an eye, which is the approximate depth of the trabecular meshwork. This should allow the needles to penetrate to a position so that their tips are close to or inside the meshwork.

As illustrated, ocular interface 110 has 16 diathermy needles 116. However, the number of diathermy needles may be varied greatly (e.g., between 2 and 300) depending on application.

Electrical signal source 120 is adapted to supply an electrical signal to diathermy needles 116. In particular implementations, the electrical signal may be of a relatively high frequency (e.g., 3 to 5 MHz). In other implementations, the signal may have other frequencies (e.g., between 1 MHz and 7 MHz). In particular implementations, the frequency of the signal supplied by electrical signal source 120 is adjustable. Electrical power source 120 is coupled to ocular interface 110 through a link 130 (e.g., a cable or power cord). Electrical signal source 110 may, for example, be a sine wave generator.

In certain modes of operation, a user (e.g., physician or other medical professional) first deadens an eye of a patient lying on a table. The deadening agent may, for example, be a topical anesthetic. The user then positions ocular interface 110 over the eye so that base 112 surrounds the cornea. The user then moves ocular interface 110 toward the eye so that diathermy needles 116 engage and bore through the eye's limbal zone. The user continues to move base 112 toward the eye (e.g., by pressing on surface 114a) until diathermy needles 116 are in or near the trabecular meshwork (e.g., until second surface 114b of ocular interface 110 engages the eye). The user may then activate electrical signal source 120 (e.g., by the use of a foot peddle). Electrical signal source 120 may deliver a preset RF signal to diathermy needles 116. The signal may be on the order of a few milliseconds in length and may be adjusted depending on application. In particular applications, multiple signals may be delivered in sequence. The entire pulse cycle may be on the order of a few seconds for a treatment.

The application of the RF signal to the trabecular meshwork causes the meshwork in the local vicinities of needles 116 to be coagulated (e.g., contracted), which results in a large portion of the meshwork being stretched and a lower resistance to fluid flow being achieved. The lower fluid flow resistance allows fluid to more easily pass (e.g., diffuse) to Schlemm's canal and a reduced eye pressure.

System 100 has a variety of features. For example, as opposed to prior art procedures, system 100 allows IOP to be treated without having to cut open portions of the eye (e.g., to access the trabecular meshwork). In fact, the thickness of the diathermy needles may qualify the system for minimal invasive treatment of glaucoma. With help of such device, the trabecular meshwork can be coagulated at a sufficient number of locations to provoke many local meshwork contractions. Overall, the contractions results in stretching of the meshwork, which leads to a reduction of its flow resistance. Therefore, an improved flow into the Schlemm's canal and a corresponding lowered IOP is obtained.

Although FIG. 1 illustrates an example system for enhancing ocular drainage, other systems for enhancing ocular drainage may include fewer, additional, and/or a different arrangement of components. For example, a system may include fewer or additional diathermy needles. As another example, an outer portion of diathermy needles may be insulated. An example insulated needle is shown in FIG. 4. The illustrated needle 116 includes an insulating sleeve 400. The sleeve 400 may be formed from Teflon or any other appropriate material with a high dielectric strength. The sleeve 400 may extend along a portion of the needle 116, with the distal end 410 of the needle 116 protruding beyond the sleeve 400. In particular implementations, only a small portion of the distal end of the needles (i.e., the ends farthest from the ocular interface) may protrude out from the insulating sleeve (e.g., less than approximately 0.1 mm in length). This may assist in focusing the application of the RF energy to enhance coagulation.

As an additional example, a spacer may be used between the ocular interface and the eye. The spacer may be particularly useful if the ocular interface is made of metal and/or to accommodate individual anatomic conditions (e.g., highly myopic or hyperopic eyes). The spacer could be made of plastic or any other appropriate material. In some implementations, a spacer may be adapted to fit inside the needles or outside the needles. In certain implementations, a spacer may include an inner piece and an outer piece with grooves at the junction to allow the needles to pass. The surface of the spacer may approximate that of the ocular interface. In particular implementations, a number of spacers may be stackable to accommodate various conditions.

An example spacer 500 is shown in FIG. 5. The spacer 500 may include a first, inner ring 510 and a second, outer ring 520. The spacer 500 may also include a central opening 530 and a plurality of apertures 540. The apertures 540 may be formed at the interface 550 between the first and second rings 510, 520. In other implementations, the spacer 500 may be a unitary piece with the apertures 540 formed therein.

The central opening 530 permits passage of the cornea through the spacer 500. The apertures 540 permit passage of the needles, such as needles 116, to pass through the spacer 500. The number of apertures 540 may correspond to the number of needles 116. The spacer 500 may also have a thickness 560. The thickness 560 may be defined to be any desired thickness.

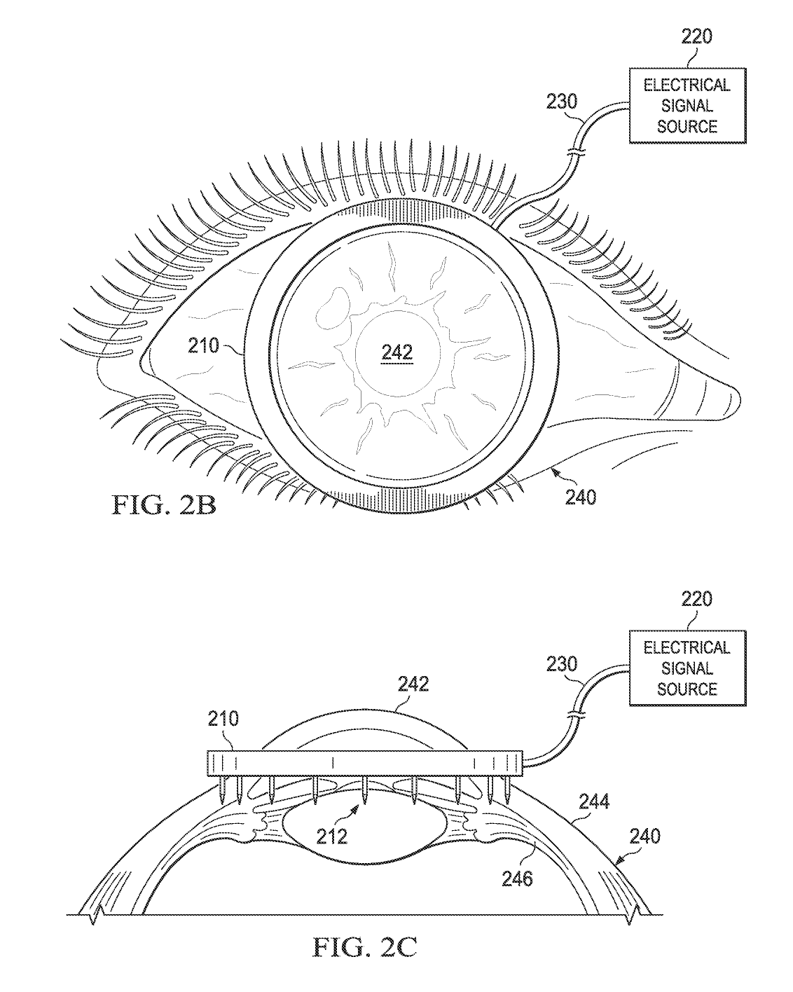

FIGS. 2A-C illustrate an example system 200 for enhancing ocular drainage in use. Similar to system 100, system 200 includes an ocular interface 210 having an annular shape and a number of diathermy needles 212 protruding therefrom, an electrical signal source 220, and a link 230 between ocular interface 210 and electrical signal source 220.

FIGS. 2A-B show ocular interface 210 positioned over an eye 240, which is shown in simplified form. As is common, eye 240 includes, among other things, a cornea 242, a sclera 244, and a trabecular meshwork 246. As illustrated, the inner portion of ocular interface 210 is wide enough so that cornea 242 may fit inside it.

In operation, a user positions ocular interface 210 over eye 240 and center the ocular interface 210 over the eye 240, as best shown in FIGS. 2A-B. The user then moves ocular interface 210 towards eye 240 so that needles 212 contact the eye 240 in the transition between cornea 242 and sclera 244 (e.g., in the limbal contour). The user then presses needles 212 through sclera 244 so that they are in or near trabecular meshwork 246, as best shown in FIG. 2C. Note that eye 240 is shown in cross section and ocular interface 210 is shown in a planer view. Thus, needles 212 do not penetrate the iris, the pupil, or the lens of eye 242, as FIG. 2C might suggest.

After seating needles 212, the user applies an RF signal through needles 212 by activating electrical signal source 220. The RF signal may, for example, be in the range of 1 to 7 MHz and may be applied for a few milliseconds. In certain implementations, a number of signals may be applied. However, most treatment sessions would require only a few seconds of treatment with the electrical signal source 220.

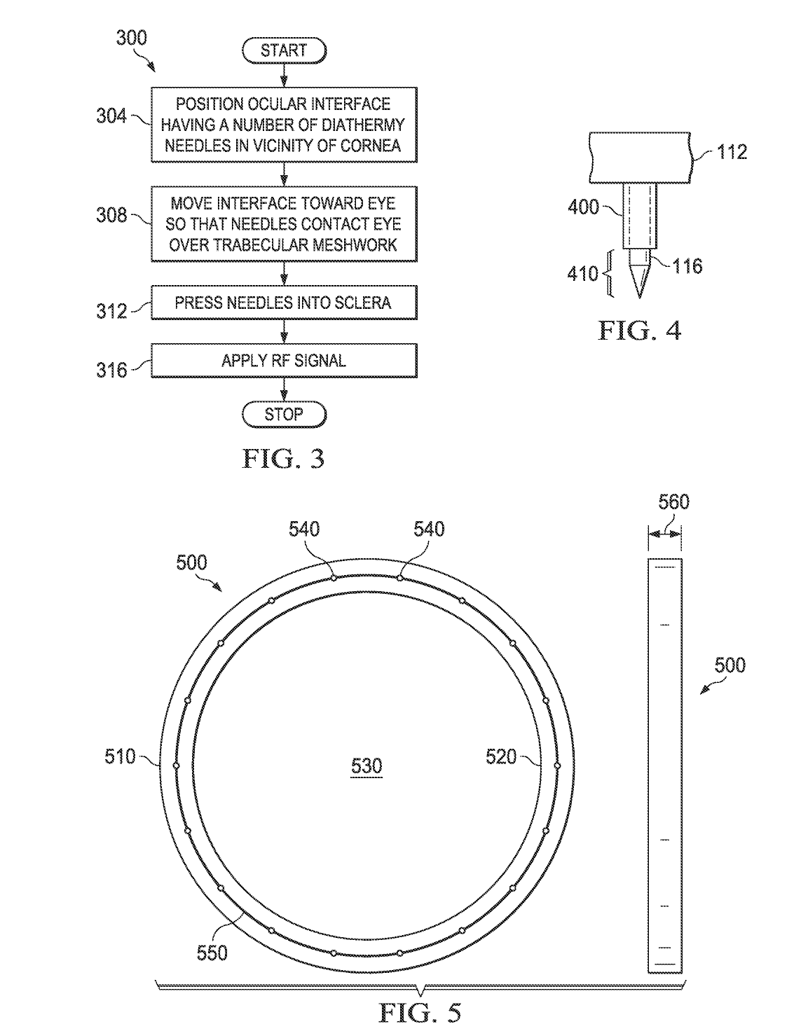

FIG. 3 illustrates an example process 300 for enhancing ocular drainage. Process 300 may, for instance, be performed using a system similar to system 100 or 200 or another system within the scope of the present disclosure.

Process 300 calls for positioning an ocular interface having a number of diathermy needles extending therefrom in the vicinity of a patient's cornea (operation 304). The ocular interface may, for example, have a base in the shape of an annular ring. The diathermy needles may range in number from 2 to 300.

Process 300 further calls for moving the interface towards the eye so that the needles contact the eye over the trabecular meshwork (operation 308). Process 300 additionally calls for pressing the needles of the device into the sclera (operation 312). The needles may be pressed into the eye until their distal tips are in or near the trabecular meshwork.

Process 300 further calls for applying an RF signal through the needles (operation 316). The RF signal may, for example, be in the range of 1 to 7 MHz and may be applied for a few milliseconds.

Although process 300 illustrates one example of a process for enhancing ocular drainage, other processes for enhancing ocular drainage may include fewer, additional, and or a different arrangement of operations. For example, a process may include performing sterilization around the eye and applying anesthetic to the eye. As another example, a process may include applying multiple RF signals to the eye. As an additional example, a process may include removing the ocular interface from the eye.

The various implementations discussed and mentioned herein have been used for illustrative purposes only. The implementations were chosen and described in order to explain the principles of the disclosure and the practical application and to allow those of ordinary skill in the art to understand the disclosure for various implementations with various modifications as are suited to the particular use contemplated. Thus, the actual physical configuration of components may vary. For example, the mentioned size(s) of components and their illustrated sizing relative to each other may vary based on application. Moreover, the shapes of one or more components may vary depending on application. Thus, the illustrative implementations should not be construed as defining the only physical size, shape, and relationship of components.

The terminology used herein is for the purpose of describing particular implementations only and is not intended to be limiting. As used herein, the singular form “a”, “an”, and “the” are intended to include the plural forms as well, unless the context clearly indicates otherwise. it will be further understood that the terms “comprises” and/or “comprising,” when used in the this specification, specify the presence of stated features, integers, steps, operations, elements, and/or components, but do not preclude the presence or addition of one or more other features, integers, steps, operations, elements, components, and/or groups therefore.

The corresponding structure, materials, acts, and equivalents of all means or steps plus function elements in the claims below are intended to include any structure, material, or act for performing the function in combination with other claimed elements as specifically claimed. The description of the present implementations has been presented for purposes of illustration and description, but is not intended to be exhaustive or limited to the implementations in the form disclosed. Many modifications and variations will be apparent to those of ordinary skill in the art without departing from the scope and spirit of the disclosure.

A number of implementations have been described for enhancing ocular drainage, and several others have been mentioned or suggested. Moreover, those skilled in the art will readily recognize that a variety of additions, deletions, modifications, and substitutions may be made to these implementations while still enhancing ocular drainage. Thus, the scope of the protected subject matter should be judged based on the following claims, which may capture one or more concepts of one or more implementations.

1. A system for enhancing ocular drainage, the system comprising:
an ocular interface comprising:
a base;
a plurality of diathermy needles extending from a side of the base, the needles extending a distance sufficient to penetrate a sclera of an eye and provide radio frequency signal energy to a trabecular meshwork therein.

2. The system of claim 1, wherein the base is an annular ring that is sized to fit around a cornea.

3. The system of claim 1, wherein the diathermy needles comprise an outer layer of insulation that extends from an end of the needles near the base to a location adjacent to a distal end of the needles.

4. The system of claim 1, further comprising an electrical signal source coupled to the ocular interface, the electrical signal source adapted to apply a high frequency radio signal to the diathermy needles.

5. The system of claim 4, wherein the electrical signal source is adapted to generate a radio frequency signal between 3 and 5 MHz.

6. The system of claim 4, wherein the electrical signal contracts the trabecular meshwork in the vicinity of the needles, which causes a large portion of the trabecular meshwork to stretch.

7. The system of claim 1, where the ocular interface comprises more than 20 diathermy needles.

8. The system of claim 1, wherein the diathermy needles are monopolar.

9. The system of claim 1, wherein the diathermy needles are bipolar.

10. A method comprising:
positioning an ocular interface comprising a base and a plurality of diathermy needles extending therefrom in the vicinity of a patient's cornea;
moving the ocular interface toward the eye so that the needles contact the eye over the trabecular meshwork; and
pressing the needles into the sclera.

11. The method of claim 9, wherein the base comprises an annular ring and further comprising centering the base over the cornea.

12. The method of claim 9, further comprising applying a radio frequency signal to the needles.

13. The system of claim 11, wherein the signal contracts the trabecular meshwork in the vicinity of the needles, which causes a large portion of the trabecular meshwork to stretch.
